(19) United States
(12) Patent Application Publication
(10) Pub. No.: US 2016/0262930 A1
(43) Pub. Date: Sep. 15, 2016

(54) DEVICE FOR SPINAL CORD COOLING AND METHOD OF SPINAL CORD INJURY TREATMENT

(71) Applicant: Robert Hansebout, Hamilton (CA)

(72) Inventor: Robert Hansebout, Hamilton (CA)

(21) Appl. No.: 14/929,859

(22) Filed: Nov. 2, 2015

Related U.S. Application Data
(60) Provisional application No. 62/132,552, filed on Mar. 13, 2015.

(51) Int. Cl. A61F 7/12

(52) U.S. Cl. CPC A61F 7/12; A61F 2007/0056

(57) ABSTRACT

The present concept is a cooling system and method for cooling the spinal cord dura which in turn cools the spinal cord. This method involves exposing the spinal cord leaving the dura intact at the location of the spinal cord injury, and applying a cooling pad to the spinal cord dura at the point of the spinal cord injury in order to cool the dura which in turn cools the spinal cord. The pad is cooled to achieve a dura temperature of between 2 to 10 degrees centigrade for a period of ½ to 96 hours. The cooling pad is a saddle shaped cooling saddle that follows the exterior profile of the dura to maximize the heat transfer between the saddle and the dura. It is made of a light soft pliable material, a biomedical grade elastomer, which conforms to the outer contour of the dura when gently placed on the dura. The cooling saddle includes an inlet and outlet for communicating cooling liquid through the pad thereby cooling the pad and the dura in contact with the pad. There are channels for communicating cooling liquid through the saddle in a preselected path from the inlet to the outlet. The inlet tube and outlet tube are each connected to a support nozzle for vertically gently suspending the saddle by the inlet and outlet tubes over the dura. Preferably a dural temperature of between 5 and 7 degrees centigrade is maintained for a period of 1 to 8 hours.

This application claims priority from the previously filed provisional application No. 62/132,552, filed on Mar. 13, 2015 by Robert Hansebout under the title: DEVICE FOR SPINAL CORD COOLING AND METHOD OF SPINAL INJURY TREATMENT.

### FIELD OF INVENTION

The present concept relates to equipment and a heat exchanger device for spinal cord cooling and a method of using the device for the treatment of severe spinal cord injuries.

### BACKGROUND

Spinal cord injury (SCI) can be devastating to the victim's quality of life. While modern treatment of SCI has lessened the associated morbidity and mortality, there remains place for improvement of outcomes. SCI has increased to approximately 50 individuals per million persons per year in North America. The main causes are motor vehicle crashes, sports, especially diving and falls. Approximately 4 males to 1 female is the generic ratio of persons affected. There are approximately 30 such injuries per day in the United States or one every 20 min in North America. In the United States alone there are approximately 10,000 such injuries per year and worldwide about one such injury every 3 min. Older persons are now becoming increasingly involved.

Complete SCI refers to a very severe injury of the spinal cord in which the spinal cord is so damaged that there is no neurological function left below the level of the lesion. Most often there is an injury to the spine as well. Incomplete neurological cord-injured persons have some degree of function, most commonly sensory function left below the area of injury. In 20% of severe injuries, the spinal cord is actually transacted so that this is also a neurologically complete injury but no recovery is possible. In approximately 80% of patients with severe SCI, the spinal cord is not divided or transacted. Such injuries can be complete or incomplete neurologically. The ratio is approximately 50 complete to 50 incomplete injuries.

There are slightly more neck injuries then thoracic spinal cord injuries. Re-hospitalizations for injured individuals are 30 to 50% per year. For medical, rehabilitative and home care the first year quadriplegic costs almost $500,000 to treat and the lifetime costs are about $1.7 million whereas a paraplegic costs about $1 million during a lifetime. There are approximately 250,000 patients with chronic spinal cord injuries in the United States.

A typical paraplegic patient with a complete thoracic SCI is confined to a wheelchair with no ability to walk, without control of bowel or bladder, no sexual function and a multitude of chest and skin complications, in addition to bladder and kidney stones and infections. The quadriplegic has similar disabilities but is additionally without hand function and some arm dysfunction, often making it difficult even to scratch their nose or feed themselves. Some require ongoing personal care supervision for the rest of their lives.

One intriguing aspect of trauma is the fact that in war or other violent environments, a bullet passing close to the spinal column can produce shockwaves to the degree that the victim can suffer a complete SCI, without the spinal cord having been touched by the bullet but only by the shockwaves.

Unfortunately, the dismal prognosis for the treatment of a severe SCI has been known since the surgical papyrus from Egypt (2500 BC) was translated by Edwin Smith. It was known then that spinal cord injured patients rarely improve. Unfortunately this belief has persisted for centuries, until about 40 years ago when some hope was shown in therapy.

In a severe SCI, the initial insult can cause a concussion or bruising of the spinal cord, the so called “primary injury” which can cause cessation of function below the lesion. However, after a severe SCI, even when the spinal cord appears initially normal and is not transacted, there begins within a few minutes a progressive auto-destructive secondary SCI sequence, the “secondary injury” which literally causes destruction of remaining spinal cord tissue from the center of the spinal cord radiating outward due to bleeding, swelling and infarction. Most of the destruction occurs within the first 8 to 12 hours after the original injury. Scientists, physicians and healthcare providers have been trying for the last 45 years to try to prevent this secondary auto-destructive mechanism using drugs such as steroids, physical agents such as whole body cooling and hyperbaric oxygen treatment.

Certainly regeneration, the ultimate treatment in patients with a chronic complete neurological SCI has been the goal of many neuroscientists for years in the laboratory and clinically. However there have been no significant beneficial results aside from the recovery of some sensation below the level of a severe injury after stem cell transplantation into the injured spinal cord.

The use of corticosteroids was considered almost mandatory treatment for a SCI 20 to 40 years ago. However, in the last two decades, their value for preserving neurological function after injury has been debated. None of the other drugs tried have shown significant benefit. Cooling of the injured spinal cord, which has been used in the laboratory for about five decades, has shown promise. We have completed a series of human complete spinal injury cases over a 10 year period. After a long term follow-up, some of the results have been very encouraging if not spectacular. We will herein described the series of patients with neurologically complete SCI, the method employed, the cooling device perfected over a decade of trials and errors and the results obtained in this series after an average follow-up of five years per patient. Our results suggest that localized cooling of the spinal cord to between 15 and 20° C. can lead to the preservation of tissue in the severely injured spinal cord leading to neurological improvement. We are also recommending that this be utilized in severe incomplete spinal-cord injured patients, since the method appears to do no harm to the spinal cord at that temperature.

### SUMMARY OF THE INVENTION

Most lay people do not realize the fragility of the human spinal cord. It is protected by the ring of bone comprising the spinal canal, by the tough dural membrane, and moves in a slow manner cushioned by the surrounding spinal fluid. During surgery, any moderate compression, shock wave close to the spinal cord or retraction for a short period of time if carried out by an inexperienced person can cause damage the spinal cord resulting in paralysis, weakness, dysfunction and other neurological complications. It is best to apply treatments such as cooling the injured spinal cord through the intact dura to prevent further damage. Our method of cooling minimizes the risk of further spinal cord damage.

The saddle-shaped heat exchanger placed on the spinal cord to cool in a transdural fashion took at least eight years to develop using various models, materials and methods. During initial development, very conductive but hard metals damaged the spinal cord. Early dural heat exchanger units were constructed to virtually encircle the cord but could compress and damage the spinal cord or the nerve roots exciting the spinal cord. After years of trial and error, experimentation and change, we discovered a method of producing a pliable light but durable cooling saddle. This rests lightly against the visible portion of dura overlying of the spinal cord. Transdural cooling using this embodiment allowed cord cooling to a temperature which averaged deep hypothermia. This degree of cooling can stop or reduce the auto-destructive sequence which damages the spinal cord after a severe injury resulting in a better neurological outcome.

The cooling apparatus consists of a box containing a reservoir for cold water and ice surrounding a module of thermoelectric units. The thermoelectric units cool a cup-shaped receptacle containing liquid on the upper surface of the cooling equipment. Liquid, between 2 and 10° C. is aspirated from the cup-shaped receptacle by suction using a Watson—Marlow peristaltic pump, which propels the water through a tube guided by an arm which can be lowered by up to 6 feet and down to 3 feet from the ground as well as being rotated 360 degrees. At the end of the arm holding the 2 tubes (one inlet and 1 outlet), are 2 tiny metal tubes or support nozzles. A cooling pad in the shape of a cooling saddle is used as a heat exchanger and is made of medical grade elastomer. Preferably a Dow Corning Silastic is used. The cooling saddle, can be positioned to lightly fit the contour of the dura over the spinal cord at the area of injury by manipulating the holding arm. The cooling saddle is contoured to fit lightly over the dura. Its size and shape fits the dura over the spinal cord in various portions of the body when accessed through either an anterior or posterior approach. Cooled liquid circulates through concentric channels in the cooling saddle to cool the spinal cord uniformly and without causing further cord harm or compression. It took years to develop a shape, contour and weight to ensure efficient rapid cooling of the cord injury and to cause no further harm to the already injured spinal cord. The cooling liquid is propelled through an inlet tube and an outlet tube along the articulating arm which can rotate, be raised and lowered so the cooling saddle sits lightly over the dura. The cooled liquid then circulates back from the heat exchanger saddle to the cup-shaped receptacle for re-cooling, then completing the circuit again. The heat exchanger saddle has a thermocouple on the dural side to measure the dural temperature.

In another embodiment, the thermoelectric units are arranged like the numbers of a clock beneath a brass plate containing tiny paths through which the cooling liquid flows and is in turn cooled by the thermoelectric units.

In both embodiments water surrounding the thermoelectric units absorbs heat radiated from the thermoelectric units and the melting ice contained in the water bath absorbs the heat. After circulating through the channels in the sterile brass plate overlying the thermoelectric units, the water is propelled through the latex tubes, described in the above embodiment above bringing cold water to the tiny malleable, saddle-shaped cooling saddle to cool the dura and back along the articulating arm into the fluid filled receptacle containing the coolant.

The original version of the apparatus uses an AC circuit to propel the peristaltic pump and energize the thermoelectric units. In the improved version, a lithium battery can be incorporated to provide electric power, with enough power to provide cooling and propulsion of liquid for 4 to 6 hours of cooling time. This will enable the system to be used in areas where AC current is not available such as in a war theater or during transport in an ambulance.

The cooling saddle has gone through at least 16 versions developed over the years. The most common unit consists of a cooling saddle containing numerous small channels through which the cold liquid passes, providing uniform cooling on the under surface of the units. The original cooling saddles were modelled to fit the posterior aspect of the dural surface. This version can be used to cool the dura over the posterior aspect of the cervical or thoracic region. Since the spinal cord varies in width in various regions, body sizes and gender, a number of different sizes of cooling saddles have been developed to enable cord cooling in both sexes and all ages in various sizes of individuals without doing more harm to the spinal cord. These cooling saddles vary in length from 11 mm to 34 Millimeters while the width can vary from 11 Millimeters to 22 Millimeters. The attachment of the inlet and outlet tubes can be perpendicular to or at an angle to the upper surface of the cooling saddle. In most instances, this cooling saddle is placed at surgery under direct vision after the dura overlying the spinal cord has been decompressed. The cooling saddle is left in place for four hours of cooling, often while the orthopedic surgeon performs a spinal fusion. It is then extracted. It can however be left in place for from an hour to several days and even with the wound closed. It can be extracted without re-opening the wound.

In many SCI cases, it has become obvious through the years that an anterior approach, especially to the anterior cervical dura and spinal cord is often more appropriate. A small circular cooling saddle has been devised which enables its passage through a minimal surgical approach via a circular access tube to rest upon the anterior portion of the dura overlying the cervical spinal cord. This small cooling saddle is specially designed to pass through a small aperture or tube during minimal invasive surgery.

All of the equipment that has any contact with the patient can be sterilized using a gas autoclave. The cooling saddles can also be sterilized in a similar fashion.

The present concept is a method of spinal cord cooling using posterior entry comprising the steps of
a) exposing the spinal cord leaving the dura intact at the location of the spinal cord injury;
b) applying a cooling pad to the spinal cord dura at the point of the spinal cord injury in order to cool the dura which in turn cools the spinal cord;
c) cooling the pad to achieve a dura temperature of between 2 to 10 degrees centigrade for a period of ½ to 96 hours.

Preferably wherein the cooling pad is a saddle shaped cooling saddle in order to follow the exterior profile of the dura to maximize the heat transfer between the saddle and the dura.

Preferably wherein the cooling saddle is made of a light soft pliable material which conforms to the outer contour of the dura when gently placed on the dura.

Preferably wherein the cooling saddle is made of a biomedical grade elastomer.

Preferably wherein the cooling saddle includes an inlet and outlet for communicating cooling liquid through the pad thereby cooling the pad and the dura in contact with the pad.

Preferably wherein the saddle includes channels for communicating cooling liquid through the saddle in a preselected path from the inlet to the outlet.

Preferably wherein the saddle includes an inlet tube and an outlet tube each connected to a support nozzle for vertically gently suspending the saddle by the inlet and outlet tubes over the dura.

Preferably wherein the cooling pad is a rounded quadrilateral in shape having a width of between 11 and 22 millimeters and a length of 11 to 34 millimeters.

Preferably wherein the cooling pad includes a thermocouple for measuring the dura surface temperature and preferably a dural temperature of between 5 and 7 degrees centigrade is maintained for a period of 1 to 8 hours.

Preferably wherein the inlet tube and the outlet tube attach to the cooling pad at an angle theta relative the pad to accommodate the spinal cord positioned on an inclined axis.

The present concept is a method of spinal cord cooling using anterior entry comprising the steps of:
a) exposing the spinal cord using a tubular entry device leaving the dura intact at the location of the spinal cord injury;
b) applying a cooling pad to the spinal cord dura at the point of the spinal cord injury in order to cool the dura which in turn cools the spinal cord;
c) cooling the pad to achieve a dura temperature of between 2 to 10 degrees centigrade for a period of ½ to 96 hours.

Preferably wherein the cooling pad is a round saddle shaped cooling saddle in order to fit through the tubular entry device and to follow the exterior profile of the dura to maximize the heat transfer between the saddle and the dura.

Preferably wherein the cooling saddle is made of a light soft pliable material which conforms to the outer contour of the dura when gently placed on the dura.

Preferably wherein the cooling saddle is made of a biomedical grade elastomer.

Preferably wherein the cooling saddle includes an inlet and outlet for communicating cooling liquid through the pad thereby cooling the pad and the dura in contact with the pad.

Preferably wherein the saddle includes channels for communicating cooling liquid through the saddle in a preselected path from the inlet to the outlet.

Preferably wherein the cooling pad includes a thermocouple for measuring the dura surface temperature and preferably a dural temperature of between 5 and 7 degrees centigrade is maintained for a period of 1 to 8 hours.

The present concept is a cooling system for cooling the spinal cord dura which in turn cools the spinal cord, the cooling system comprising;
a) a saddle shaped cooling saddle which can be lightly rested onto the exterior profile of the spinal dura to maximize the heat transfer between the saddle and the dura,
b) the cooling saddle includes an inlet and outlet and at least one channel for communicating cooling liquid through the saddle thereby cooling the saddle and the dura in contact with the saddle

Preferably wherein the saddle includes an inlet tube and an outlet tube connected to the inlet and outlet respectively, for communicating cooling liquid to and from the saddle, each tube is connected to a support nozzle for vertically gently suspending the saddle by the inlet and outlet tubes over the dura.

Preferably further includes an articulating arm and a cooling unit, the articulating arm attached to the support nozzles and communicating cooling fluid between the support nozzles and the cooling unit, the cooling unit for cooling the cooling liquid to a preselected temperature.

Preferably wherein the cooling saddle is made of a light soft pliable material which conforms to the outer contour of the dura when gently placed on the dura.

Preferably wherein the cooling saddle is made of a biomedical grade elastomer.

Preferably wherein the cooling saddle is a rounded quadrilateral in shape having a width of between 11 and 22 millimeters and a length of 11 to 34 millimeters.

Preferably wherein the cooling saddle includes a thermocouple for measuring the dura surface temperature and preferably a dural temperature of between 5 and 7 degrees centigrade is maintained for a period of 1 to 8 hours.

Preferably wherein the inlet tube and the outlet tube attach to the cooling saddle at an angle theta relative the cooling saddle to accommodate the spinal cord positioned on an inclined axis.

Preferably wherein the cooling saddle is a round saddle shaped cooling saddle in order to fit through a tubular entry device and saddle shaped to follow the exterior profile of the dura to maximize the heat transfer between the saddle and the dura.

Preferably wherein the cooling saddle weighs between 1 and 6 grams to avoid damage to the spinal cord.

Preferably wherein the cooling saddle weighs between 2 and 4 grams to avoid damage to the spinal cord.

### BRIEF DESCRIPTION OF THE DRAWINGS

The present concept will now be described by way of example only with reference to the following drawings in which:

FIG. 1 is a schematic view depicting a method and system for cooling the spinal cord dura.

FIG. 2 is a schematic bottom plan view of a cooling saddle used for placement onto the dura at the injured area.

FIG. 3 is a schematic side cross sectional view of the cooling saddle used for placement onto the dura at the injured area.

FIG. 4 is a schematic end cross sectional view of the cooling saddle used for placement onto the dura at the injured area.

FIG. 5 is a partial cutaway schematic perspective view of a cooling saddle used for placement onto the dura of a spinal cord at the injured area.

FIG. 6 is a schematic perspective view of a round cooling saddle used for anterior entry with a tubular entry device.

FIG. 7 is a schematic perspective view of a round cooling saddle used for anterior entry installed through a tubular entry device shown deployed in a person shown in dashed lines.

FIG. 8 is a table of Indications for Spinal Cooling

FIG. 9 is a table of Steps for Preoperative Evaluation of the Patient

FIG. 10 is a table of the steps of an Operation

FIG. 11 is schematic of cooling saddle used in posterior entry.

FIG. 12 is a schematic of a posterior entry cooling saddle positioned onto a dura of the spine.

FIG. 13 is schematic of an inclined cooling saddle used in posterior entry.

FIG. 14 is a schematic of a posterior entry inclined cooling saddle positioned onto a dura of the spine.

FIG. 15 is a schematic representation of a cooling saddle suspended from an articulating arm ready for placement onto the dura of a spinal cord.

### DETAILED DESCRIPTION OF A PREFERRED EMBODIMENT

The dosing regimen of dexamethasone used here was developed using information which showed that its administration preserved intracellular potassium, reduced tissue damage, and was correlated with better neurological recovery. We administered steroids to our patients for 11 days with gradual tapering to cessation on the 18th day.

### Cooling After SCI

There has been interest in cooling the injured spinal cord to enhance recovery of neurological function. Currently there are centers in which systemic total body cooling is used after SCI, however the temperatures achieved are only a few degrees less than normal body temperatures.

We felt that spinal cord cooling could be beneficial in the treatment of the injured spinal cord but that the optimal cooling temperature might be lower than what is feasible through systemic hypothermia. We elected to use local hypothermia, allowing for more profound selective cooling of injured tissue while avoiding potential complications of deep general hypothermia. We opted for localized extradural cord hypothermia using equipment described herein. Combining steroid agents and local spinal cord cooling at a central cord temperature of around 17° C. produces better neurological motor recovery than conservative treatment or the single use of either.

### Equipment

We began using spinal cord cooling in patients. An active study lasted 10 years, and the mean follow-up period was 4.9 years (range 14-153 months see Table 1. The original cooling apparatus was large and cumbersome. A light, portable unit that could be deployed in various hospitals was later built.

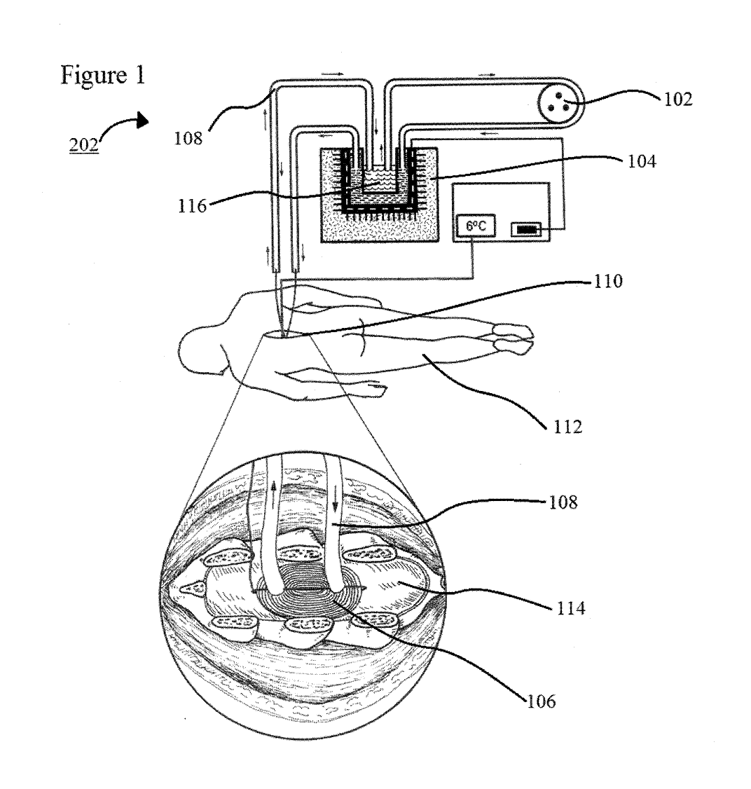

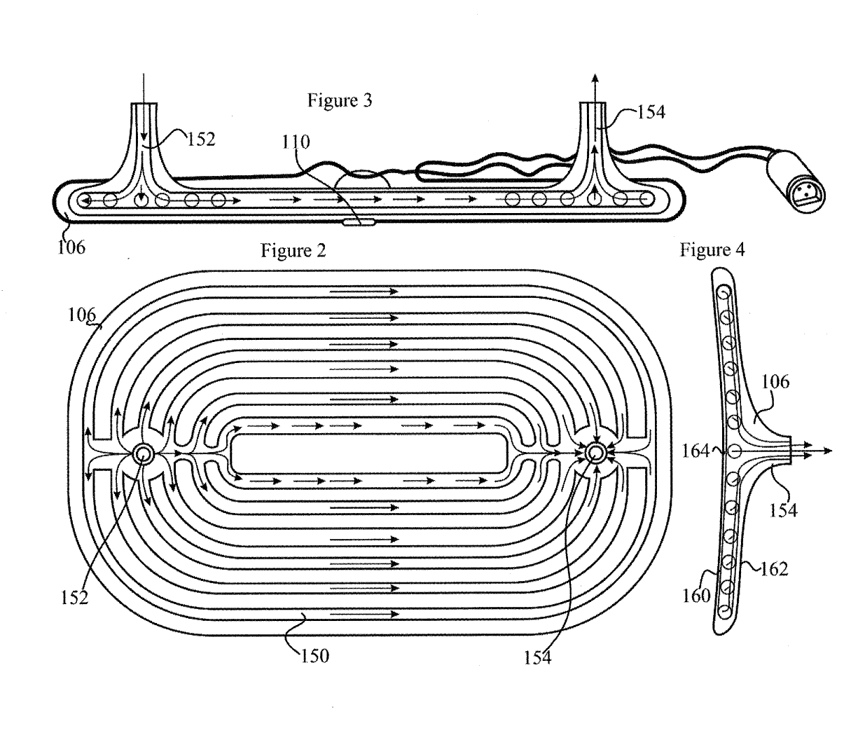

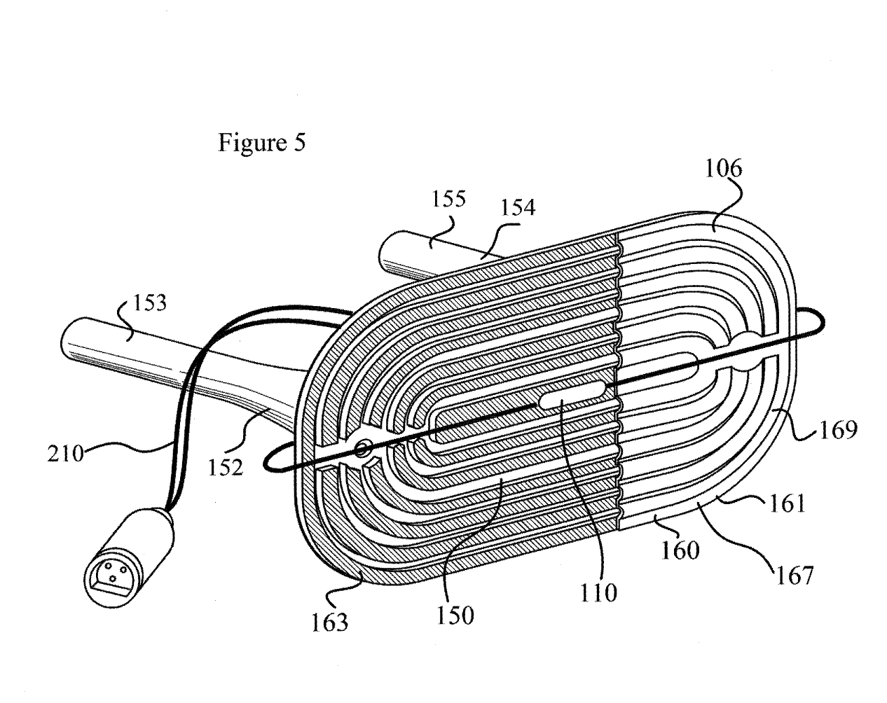

The cooling system and method of spinal cord cooling is schematically depicted in FIG. 1 and includes a cooling unit 202 with a heat exchanger 104 which communicates cooling liquid with a cooling saddle 106. A rotary pump 102 propels cooling liquid 108 which is normally saline solution through heat exchanger 104 which cools the cooling liquid to 3° C., and pumps it via rotary pump 102 to a cooling saddle 106. Cooling pad 167 is preferably a cooling saddle 106 depicted in FIGS. 2, 3,4 and 5 and includes an inlet 152, and outlet, 154, a thermocouple 110, a bottom 160, a top 162, a bottom surface 169 a center line 164 and multiple microscopic concentric channels 150 through which the cooling liquid flows. Saddle 106 includes a bottom cover 161 shown partially removed in FIG. 5 revealing channel walls 163 and cooling liquid channels 150. The cooled saline 108 enters at inlet 152 travels through channels 150 and exits at outlet 154. The bottom surface 169 of bottom cover 161 of cooling saddle 106 is rested lightly upon the unopened dura 114. Saddle 106 conforms to the shape of the dura since it is made of a soft pliable elastomeric material. Cooling liquid 108 is used to cool the dura to 6° C. as measured by thermocouple 110 in contact with the spinal dura. Thermocouple 110 measures the dural temperature. Cooling liquid 108 is then returned from the cooling saddle 106 to reservoir 116, completing the closed circuit. The cooling system is diagrammatically represented in FIG. 1 and the saddle is depicted in FIGS. 2, 3, 4 and 5.

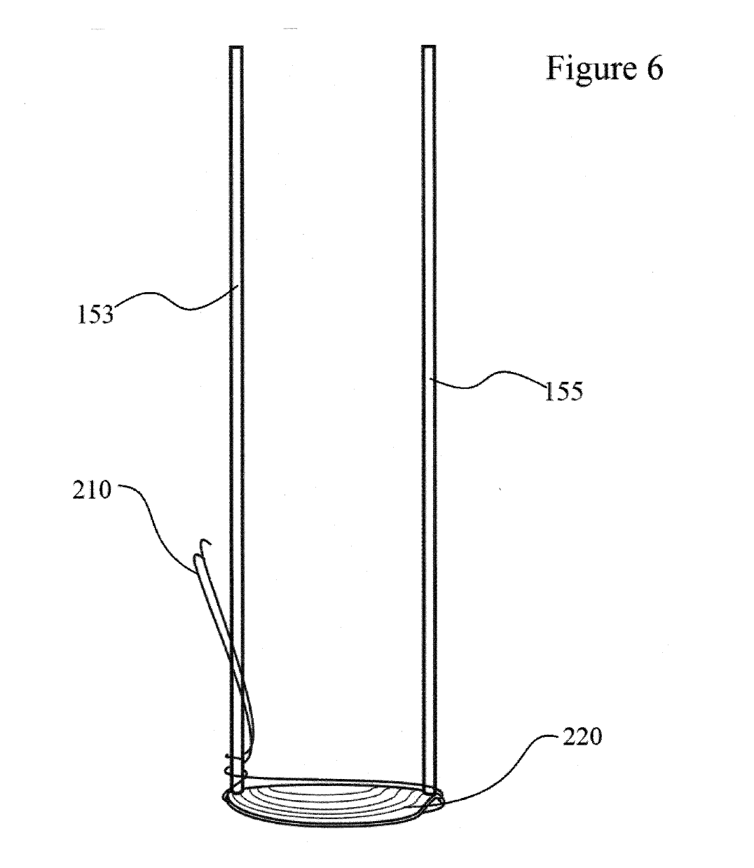

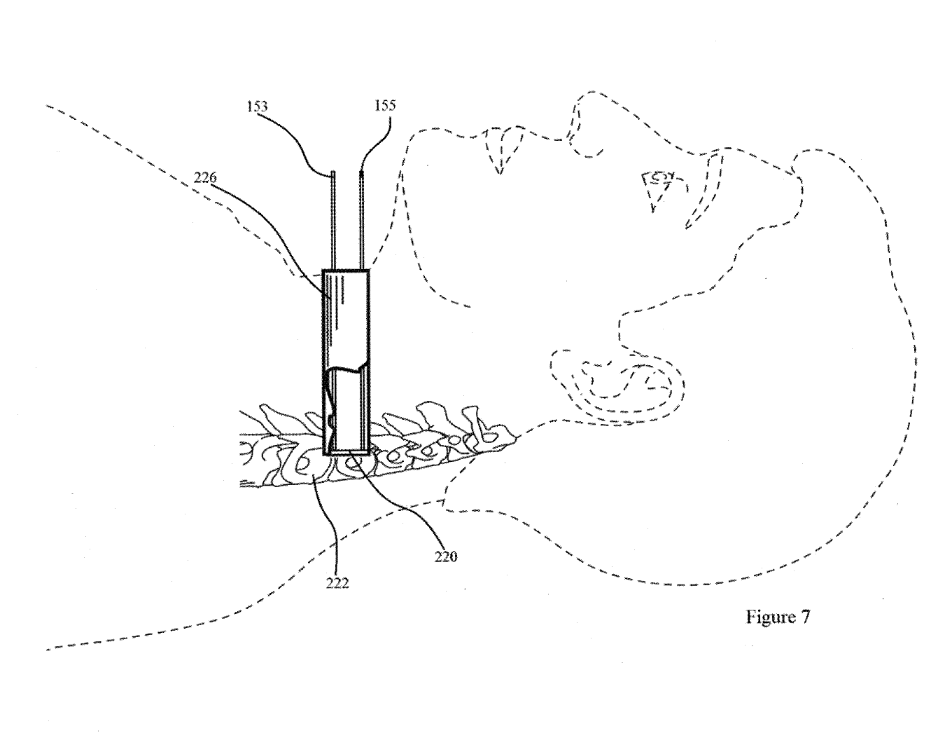

Referring to FIGS. 6 and 7 cooling saddle 220 is a round shaped saddle that fits within the inner diameter of tubular entry device 226. Tubular entry device 226 is preferably used for anterior entry to the anterior side of the spinal cord. FIG. 7 shows cooling saddle 220 deployed anteriorly to the spine 222 of a person shown in dashed lines.

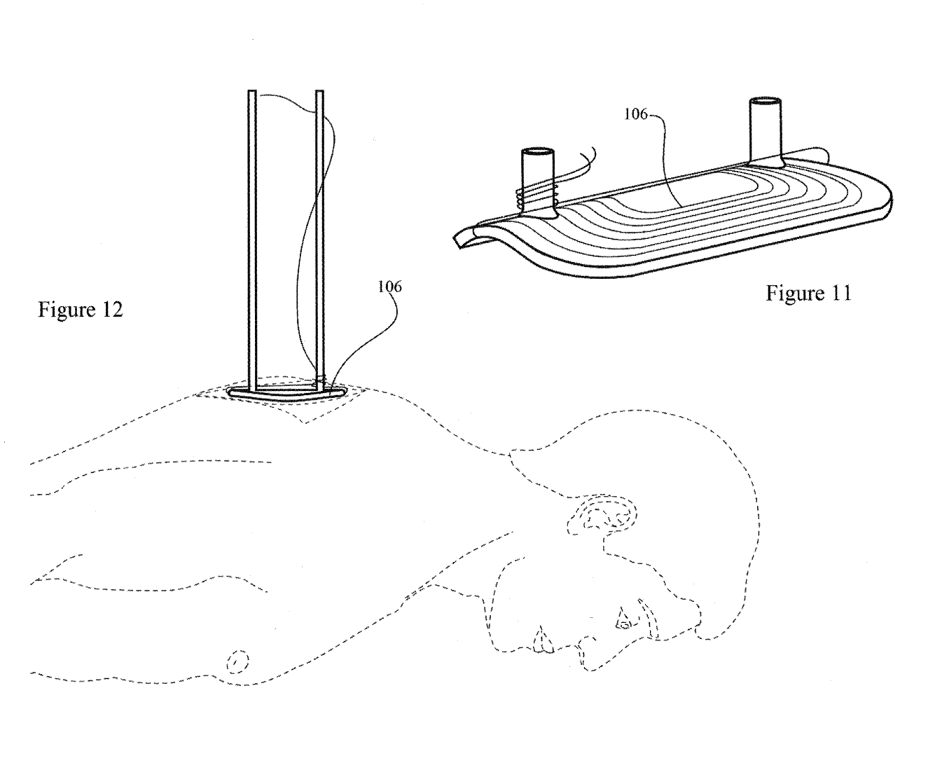

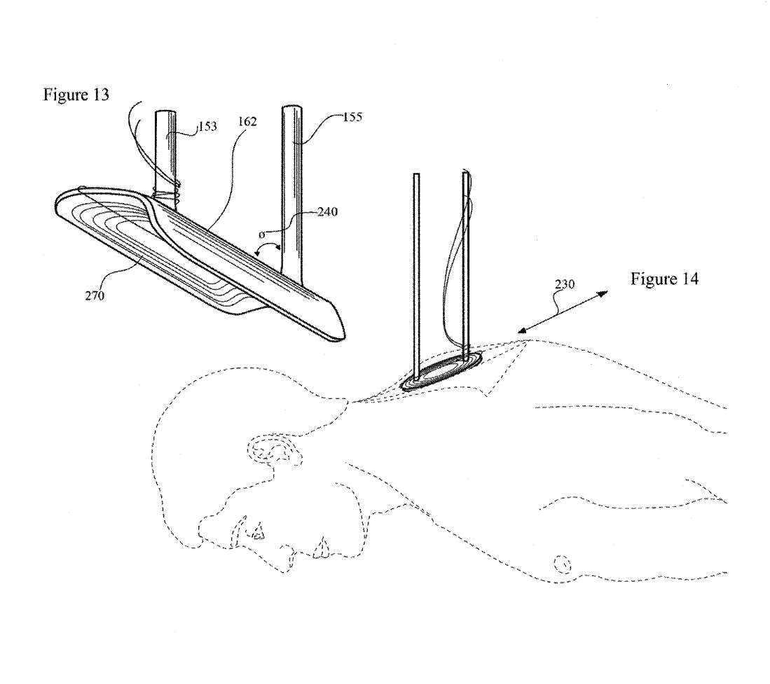

Referring now to FIGS. 11 and 12 a cooling saddle 106 is schematically depicted deployed onto a dura of a patient shown in dashed lines. In FIGS. 13 and 14 in cases were the spine is oriented at an inclined axis 230 the inlet tube 153 and outlet tube 155 are oriented at an angle theta 240 relative the top 162 of cooling saddle 270 such that the bottom surface 169 of cooling saddle 270 matches the contour of the inclined spine along inclined axis 230 when suspended by the inlet and outlet tubes 153 and 155.

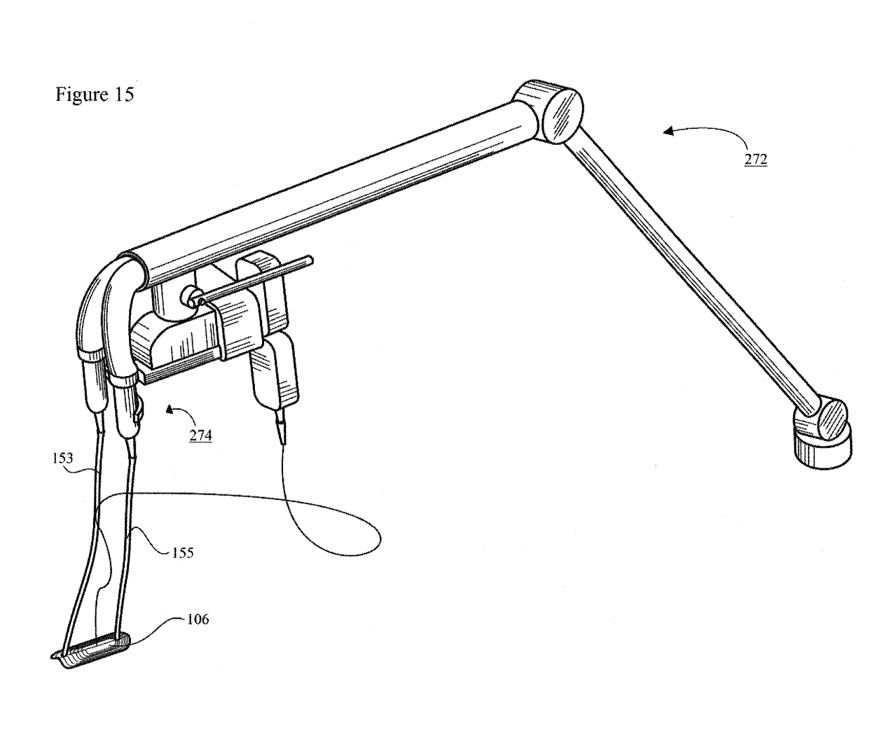

FIG. 15 depicts schematically an articulating arm 272 which communicates cooling liquid 108 from support nozzles 274 at one end to the other end of the articulating arm internally and ultimately to the cooling unit 202. Support nozzles 274 connect with the inlet and outlet tubes 153 and 155 thereby suspending the cooling saddle 106 by the inlet and outlet tubes 153 and 155. Articulating arm 272 can manipulate the position of cooling saddle 106 to any desired location thereby making it possible to gently place cooling saddle 106 upon the surface of the dura.

TABLE 1
Summary of patient characteristics

| | | | | | | | | Hrs | | Last | ASIA |
|---|---|---|---|---|---|---|---|---|---|---|---|
| | | | | | | | Hrs | After | | Exam | Score |
| | Age | | | | | | After | Injury | | After | at |
| Case | (yrs). | Injury | | | Levels | Levels | Injury | to | Hrs | Injury | Last |
| No. | Sex | Site | Cause | Force | Compressed | Fused | to Op | cooling | Cooled | (mos) | Exam |
| 1 | 21, M | T | struck | FL | T12-L1 | T10- | 5 | 6 | 4 | 52 | C |
| | | | | | | L3 | | | | | |
| 2 | 24, M | C | Fall | FL | C4-5 | C4-5 | 4.2 | 5.5 | 1.25 | 14 | C |
| 3 | 24, M | C | MVC | FL | C-6 | C5-7 | 9 | 10.5 | 3 | 84 | B |
| 4 | 36, M | C | Dive | AL | C4-5 | C3-6 | 5.25 | 6.5 | 1.5 | 54 | D |
| 5 | 38, F | C | MVC | FL | C-4 | C3-5 | 4 | 5.5 | 4 | 33 | A |
| 6 | 26, M | C | MVC | FL | C-5 | C5-6 | 5 | 7 | 4 | 71 | B |
| 7 | 64, F | T | Fall | AL | T9-10 | T6-12 | 6.5 | 7.7 | 4 | 45 | A |
| 8 | 17, F | C | MVC | AL | C5-6 | C4-7 | 5 | 7 | 4 | 50 | A |
| 9 | 51, F | T | Struck | AL | T-4 | T2-6 | 11 | 12 | 4 | 86 | A |
| 10 | 23, M | C | Dive | AL | C-4 | C3-5 | 5.5 | 7.5 | 4 | 84 | B |
| 11 | 21, F | C | MVC | FL | C-6 | C5-6 | 6 | 7 | 4 | 60 | A |
| 12 | 19, M | C | MVC | FL | C4-5 | C4-6 | 4 | 8 | 4 | 60 | B |
| 13 | 24, M | T | Fall | FL | T-6 | T4-8 | 7 | 9 | 4 | 81 | D |
| 14 | 21, M | C | MVC | FL | C-4 | C3-6 | 6 | 7.5 | 4 | 75 | C |
| 15 | 14, M | C | Dive | AL | C-5 | C4-6 | 4 | 6 | 4 | 84 | B |
| 16 | 16, M | C | Struck | AL | C-5 | C4-6 | 2.25 | 3.25 | 4 | 18 | A |
| 17 | 18, M | C | MVC | FL | C-7 | C5-T1 | 7.5 | 7.7 | 4 | 72 | C |
| 18 | 19, M | T | Fall | AL | T-7 | T5-10 | 6 | 7 | 4 | 153 | B |
| 19 | 34, M | T | Fall | FL | T9-10 | T8-13 | 3.5 | 5 | 4 | 33 | C |
| 20 | 20, F | C | Fall | FL | C4-5 | C4-5 | 6 | 7.2 | 4 | 14 | A |

* AL = axial loading injury;
C = cervical spine;
FL = flexion injury;
MVC = motor vehicle crash;
T = thoracic spine.

### Protocol

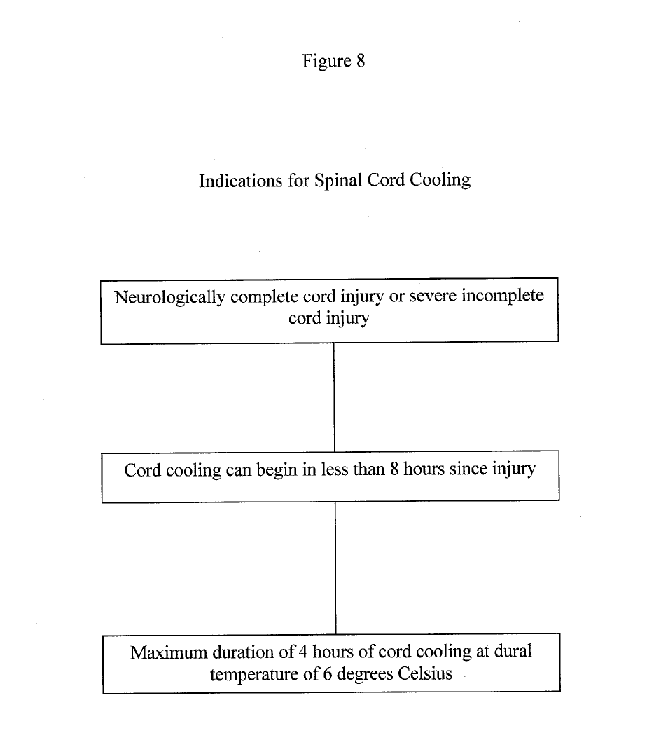

A protocol was set up at hospitals in our catchment area in Canada to inform the inventor when a patient with a severe SCI was to be transported to the trauma hospital. It was suggested to the referring physician that a loading dose of 20 mg of dexamethasone be given intramuscularly before rapid transfer. The inventor examined patients to determine neurological completeness and to gauge the patient's suitability for inclusion in the series. Eligibility criteria required that patients be alert and cooperative; between the ages of 16 and 65 years; and have no motor or sensory function below the level of cord injury, no perianal sensation, and no anal sphincter contraction. Cooling was offered only to patients with a clinically complete cord injury. It is believed that a person with a neurologically “complete” SCI rarely recovers function below the lesioned level when conventional treatment is applied. We made a diagnosis of a “complete spinal cord injury” based on the finding of completely absent neurological function. Because the patients in our series had no bulbocavernosus reflex, an absolute diagnosis of complete injury could not be made due to the possibility of spinal shock. Allowing time for recovery of the reflex, however, would have meant delaying treatment to the point where it would have lost substantial efficacy. We aimed to begin cooling the cord within 8 hours of injury. Individuals fitting the selection criteria constituted less than 3% of patients with a severe SCI seen in our trauma hospital, and consequently the series took 10 years to acquire the 20 patients. FIG. 8 summarizes the indications which are required for application of spinal cord cooling.

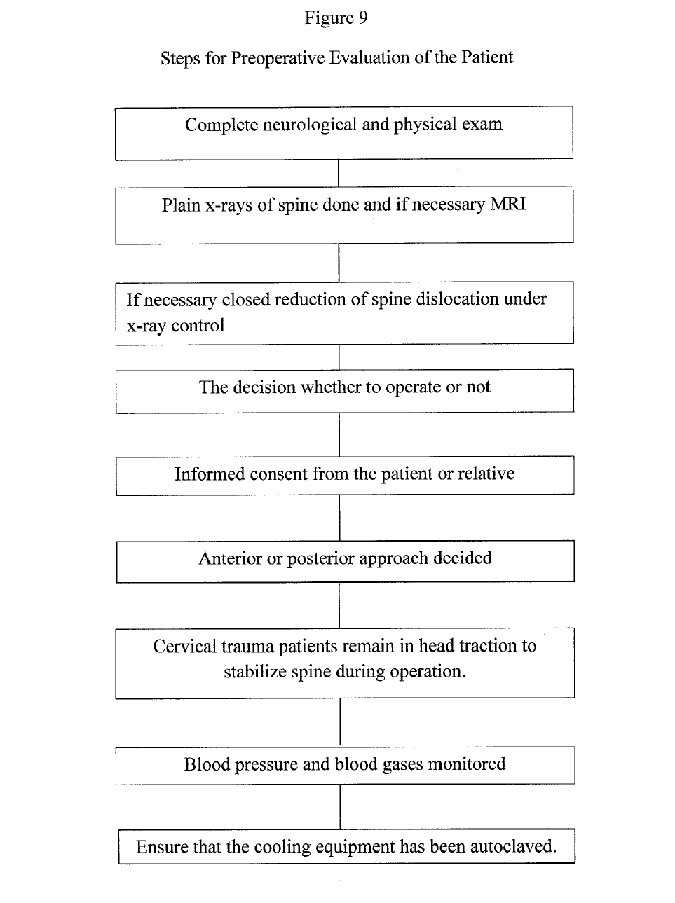

The nature of the procedure was explained and full informed consent obtained. During investigation, the patient's blood pressure and blood gases were monitored and maintained within an acceptable range. In cases of cervical injuries, halo traction was applied. The patient's shoulders were pulled down to improve visualization on radiographs of the cervical vertebrae. If necessary, closed reduction was tried during traction under radiographic control. In the interest of time, plain radiographs (3 high-quality views) were the only images taken at the injury site since the dura and occasionally the cord through a disrupted dura would be visualized at surgery. An anterior or posterior operative approach was decided on in collaboration with an orthopedic surgeon. In cases of cervical trauma, the patient remained in traction during surgery. FIG. 9 summarizes the preoperative evaluation steps of a patient.

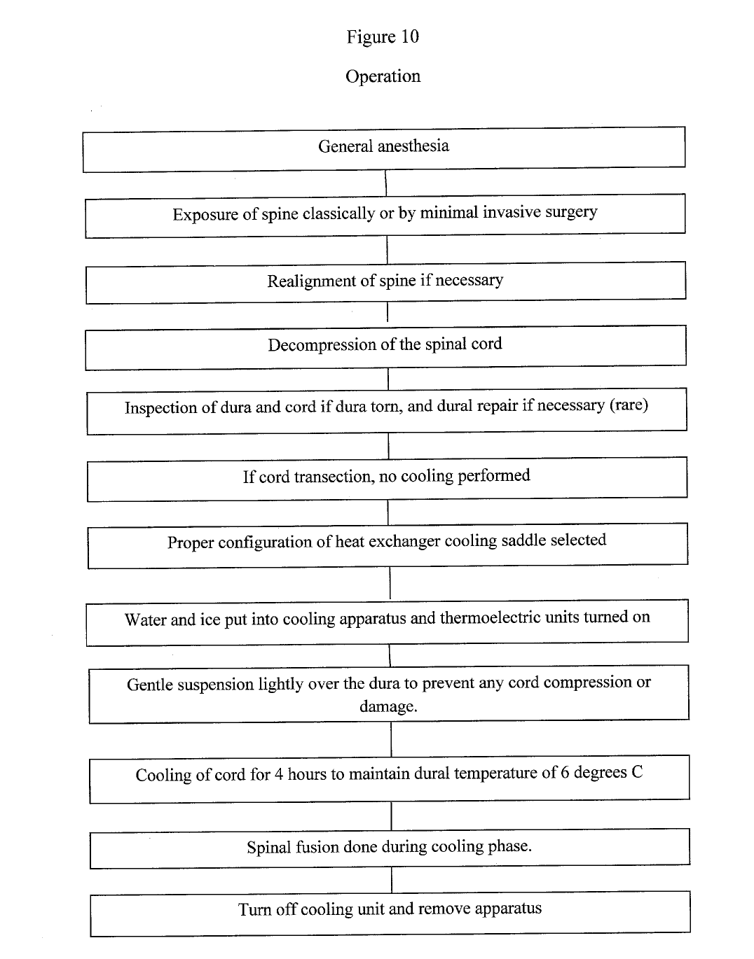

FIG. 10 summarizes the operative steps of a patient. Following exposure, any necessary vertebral alignment was completed, and the spinal cord was decompressed. The suspended cooling saddle 106 was then placed lightly against the dura 114. Transdural cord cooling was continued for up to 4 hours at a dural temperature of 6° C. Fusion was often performed by the orthopedic surgeon during the cooling stage. On cessation of cooling, the dura 114 and cord were allowed to warm spontaneously. Routine postoperative care was conducted, including intermittent bladder catheterization and a strong emphasis on chest physiotherapy. Dexamethasone was given according to a dosing regimen developed from our earlier laboratory studies: 6 mg every 6 hours was given for 11 days and then tapered gradually until discontinuation on the 18th day after injury.

The patients were seen and examined by the inventor and a spinal cord team during hospitalization and later in clinics. Information was recorded and included any additional injury or complication. Complications were further classified as “early,” if they occurred within the first month after injury, or “late,” if they occurred later. Neurological evaluation of motor and sensory recovery was performed by the inventor at intervals during the follow-up, which varied from 14 to 153 months and was, on average, 4.9 years after the injury see Table 1.

We had used our own 5-point scale to measure sensory and motor function initially and later for determination of any improvement. Our motor power grades resembled the usual grades for strength and are comparable to those found in the International Standards for the Neurological Classification of Spinal Cord Injury (ISNCSCI) and used to classify patients according to the ASIA Impairment Scale. Our sensory scale had 6 grades, corresponding to 0 (no sensation), 1 (crude pressure), 2 (touch sensation), 3 (pain and temperature or dorsal column sensation), 4 (subnormal pain, temperature and dorsal column sensation), or 5 (normal sensation). In view of the popularity of the ASIA grading scale, we matched our data to the 2011 modifications of the ISNCSCI. Our Grade 0 remained the same; we combined our Grades 1-4 and converted them to Grade 1 on the ISNCSCI sensory scale. Our Grade 5 was converted into Grade 2 sensation on the ISNCSCI sensory scale. Determination of motor level, sensory level, neurological level of injury (NLI), and zone of partial preservation were as per Kirshblum et al. Briefly, to quantitate the degree of neurological recovery, particularly of the upper extremities, we determined the single NLI. The NLI refers to the most caudal segment of the spinal cord with normal (Grade 2/2) sensory and antigravity (Grade 3/5) motor function, provided that there is normal sensory and motor function rostrally. This may differ between left and right sides of the body. Therefore, 4 separate levels are possible: a right sensory level; left sensory level; right motor level; and a left motor level. The single NLI is the most rostral of these 4 levels. We determined the motor level for each side, which indicates ASIA Grade 3 motor power muscle function provided there is normal (Grade 5/5) motor power at the rostral level. A higher score can reflect improvement in function of the extremities. In addition, we also determined the sensory level, the most caudal neurological level with ASIA sensory Grade 2 (normal sensation). Return of sensation to normal can provide protection from skin breakdown, detect bladder fullness, general sensation, sexual sensation, and allow better balance and proprioception. These measurements were determined in the acute stage. We later compared these findings with those determined on follow-up.

The ability of patients to complete various activities of daily living and enjoy some degree of mobility was recorded. Should they have had sufficient function to perform these tasks, as well as to have attested to their ability to live independently, they were considered to be self-sufficient. We also inquired about bowel and bladder control as well as sexual function.

### Results

Three patients underwent treatment but were not included in the series. One of these had a severe head injury and died within 2 weeks. A second patient had an incomplete SCI but was treated very early on. He is the only patient with an incomplete SCI who underwent decompression, cooling, and steroid therapy; he made a remarkable and rapid recovery from a severe, though incomplete, cord injury. A third patient did have a neurologically complete SCI but another surgeon treated him. The patient recovered quite well, but recorded data were incomplete. These three were all the individuals excluded from the series.

Four of our 20 patients shown in Table 1 received treatment at the Montreal Neurological Hospital; the remainder were treated at the Hamilton General Hospital. There were 14 males and 6 females. Patients were followed up for at least 1 year. All 20 patients presented with complete neurological SCI. Of these, 14 had cervical injuries while 6 had thoracic injuries. Motor vehicle crashes caused 8 injuries, 6 were caused by falls, 3 resulted from diving, and 3 were caused by a forceful blow. A flexion force to the spine caused injury in 12 cases, while axial loading caused injury in 8. The average age of the males in the series was 22.8 years, and the average age of the females in the series was 35.1 years, reflecting the presence of 3 women aged 64, 51, and 38 years. Some individuals were followed up for as long as 12 years; the average follow-up period was just less than 5 years. Eight (40%) of the patients had sustained multiple injuries, and 2 (10%) had suffered a significant head injury as summarized in Table 2.

TABLE 2
Factors having a deleterious effect on recovery

| Factor | No. of Patients (%) |
|---|---|
| Head Injury | 2 (10) |
| Multiple Injuries | 8 (40) |
| Early Complications | 16 (80) |
| Late Complications | 18 (90) |

All patients received decompressive surgery, parenteral steroids, and local spinal cord cooling. Table 3 shows the average time after injury for steroid administration, the time between injury to the start of cooling, and total duration of cooling. As dexamethasone was often administered at collector hospitals, it was delivered relatively early (mean 5.6 hours post-injury). Due to patient transfer, medical stabilization, and surgical exposure of the dura 114, cooling was begun on average 7.1 hours post-injury. We aimed for the duration of cooling to be 4 hours. The average duration of cooling was 3.7 hours.

TABLE 3
Mean timing of various interventions

| Intervention | Time in Hrs |
|---|---|
| Time to Steroid Administration | 5.6 |
| Time to cooling start | 7.1 |
| Duration of cord cooling | 3.7 |

At the onset of cooling, the dura often showed reddish coloration due to hemorrhage from the underlying injured cord and the dura was tightly stretched over the cord. After several hours of cooling at 6° C., the dura was lighter in coloration and appeared to be less tightly stretched over the cord, although dural pulsations were not observed.

A majority of patients required additional surgery, often tracheostomy. Decubitus ulcer debridement or pyloroplasty was rarely required. Early complications were pulmonary related and included atelectasis and pneumonia, followed by decubitus ulceration, spinal instability, gastrointestinal bleeding, depression, and deep vein thrombosisas summarized in Table 4. Late-onset complications were decubitus ulceration, urinary tract infection, bladder calculi, and pain.

TABLE 4
Summary of complications*

| Complication | No. of Patients |
|---|---|
| Early Onset: | |
| atelectasis | 9 |
| pneumonia | 7 |
| debucitus ulcers | 6 |
| Bone instability | 5 |
| GI Bleeding | 4 |
| Depression | 4 |
| Deep vein thrombosis | 4 |
| Cardiac arrest | 2 |
| Increased neural deficit | 2 |
| CSF leaf | 2 |
| Septicemia | 2 |
| Postoperative infection | 1 |
| Hydrocephalus | 1 |
| Meningitis | 1 |
| Late Onset: | |
| Decubitus ulcers | 10 |
| urinary tract infection | 6 |
| Bladder stones | 5 |
| Pain | 4 |
| Respiratory | 3 |
| Deep vein thrombosis | 1 |
| Spasticity | 1 |

*An early-onset complication was defined as one occurring within 1 month of injury and a late-onset complication as one occurring more than 1 month after injury. GI = gastrointestinal.

Impairment in all patients was initially categorized as ASIA Grade A. Final follow-up ASIA grades are summarized in Table 5. Impairment in 7 patients (35%) remained ASIA Grade A, improved in 6 (30%) to Grade B, improved in 5 (25%) to Grade C, and improved in 2 (10%) to Grade D. Of the 14 patients with cervical injuries, the status of 5 (35.7%) remained ASIA Grade A; it improved in 5 (35.7%) to Grade B, improved in 3 (21.4%) to Grade C, and improved in 1 (7.2%) to Grade D. The remaining 6 patients sustained a complete thoracic cord injury: 2 (33.3%) remained Grade A, 1 (16.7%) improved to Grade B, 2 (33.3%) improved to Grade C, and 1 (16.7%) improved to Grade D.

TABLE 5
Summary of final follow-up ASIA grades*

| | No. of Patients w/SCI (%) | | |
|---|---|---|---|
| Final Grade | Cervical | Thoracic | All Patients |
| A | 5 (35.7) | 2 (33.3) | 7 (35) |
| B | 5 (35.7) | 1 (16.7) | 6 (30) |
| C | 3 (21.4) | 2 (33.3) | 5 (25) |
| D | 1 (7.2) | 1 (16.7) | 2 (10) |
| Total | 14 (100) | 6 (100) | 20 (100) |

*All patients initially had ASIA Grade A impairment.

Improvement in the NLI signifies improvement in sensorimotor function in the patients. Taken as a group, our patients experienced a caudal descent of 21 vertebral segments for a mean of 1.05 segmental levels per patient as summarized in Table 6. In the 14 patients with cervical injury, there was caudal improvement in the NLI of 13 vertebral segmental levels, a mean of 0.93 levels per patient. For patients with thoracic injury, there was improvement of 8 segmental levels, a mean of 1.33 levels per patient.

TABLE 6
Caudal improvement in level of neurological function*

| | Neurological | | Motor Level | | Sensory Level | | |
|---|---|---|---|---|---|---|---|
| | Level of Injury | | to Grade ≧3 | | to Grade 2 | | Total |
| Level of | Segmental | Mean | Segmental | Mean | Segmental | Mean | No. |
| Spinal | Levels | Segmental | Levels | Segmental | Levels | Segmental | of |
| Injury | Recovered | Levels/Pt | Recovered | Levels/Pt | Recovered | Levels/Pt | Pts. |
| Cervical | 13 | 0.93 | 13 | 0.92 | 43.5 | 3.11 | 14 |
| Thoracic | 8 | 1.33 | 15 | 2.5 | 12 | 2 | 6 |
| All | 21 | 1.05 | 28 | 1.73 | 55.5 | 2.77 | 20 |

*Pt = patient.

A descent of the motor level signifies improvement in motor function, especially in the extremities. We compared our motor level findings after initial injury with those on follow-up to determine improvement in motor function. As a group, there was a caudal migration of the motor level by 28 levels, a mean of 1.7 functional levels per patient. In the patients with cervical injury, 13 levels were recovered for a mean of 0.92 levels per patient. Paraplegic patients with thoracic injuries had a return of 15 levels for a mean of 2.5 levels per patient.

The sensory level is the most caudal dermatome at and rostral to which there is bilaterally normal sensation. Return of sensation to normal can provide protection from skin breakdown, allow the detection of bladder fullness, and provide for sexual sensation. Caudal migration of the sensory level in patients with cervical injury can provide much useful function, especially in the hands. For all 20 patients, there was a caudal migration of the sensory level of 55.5 dermatomal levels for a mean of 2.8 segmental levels per patient. For patients with cervical injury, 43.5 levels were recovered, a mean of 3.1 levels per patient. For patients with thoracic injury, 12 levels were recovered, a mean of 2 levels per patient as summarized in Table 6.

There are various grades of return of motor and sensory function. So far we have not taken into consideration the total return of all the motor and sensory function in any one patient, including motor function below Grade 3 and sensory function of Grade 1 seen in the zones of partial preservation. To have a picture of motor and sensory improvement in all patients, the total motor scores and total sensory scores were tallied for each patient on admission and on the last follow-up to create a motor index score and sensory index score shown in Table 7. The total motor index score showed a dramatic increase of 174% in the patients with cervical injury, whereas the increase was only 21% in the patients with thoracic injury. In all patients, the percentage increase in the motor index score was 63%. Similarly, the sensory index score increased 167% in patients with cervical injury, reflecting the increased sensation on follow-up compared with admission. There were also increases in the sensory index score by 32% and 88% in the thoracic spine-injured patients and all patients, respectively. The mean motor scores for all patients increased from 20.8 to 34 from admission to final follow-up. The mean sensory scores increased from 59.8 to 112.6 at the same time points. In patients with cervical injury, the mean motor scores rose from 8.3 to 22.6 and the mean sensory scores from 35.7 to 95.4 from admission to final follow-up. The mean motor scores for thoracic spine-injured patients rose from 50 to 60.5, while the mean sensory score rose from 116 to 152.7 from admission to last follow-up, respectively. These figures reflect a considerable return of motor and sensory function.

TABLE 7
Improvement in motor and sensory scores*

| Level of | Motor Index Scores | | | | | Sensory Index Scores | | | | |
|---|---|---|---|---|---|---|---|---|---|---|
| Spinal | Admission | Admission | FU | FU | % | Admission | Admission | FU | FU | % |
| Injury | (total) | (mean) | (total) | (mean) | Increase | (total) | (mean) | (total) | (mean) | Increase |
| Cervical | 116 | 8.3 | 318 | 22.6 | 174 | 500 | 35.7 | 1336 | 95.4 | 167 |
| Thoracic | 300 | 50 | 363 | 60.5 | 21 | 696 | 116 | 916 | 152.7 | 32 |
| All | 416 | 20.8 | 680 | 34 | 63 | 1196 | 59.8 | 2252 | 112.6 | 88 |

*FU = follow-up.

At the time of final follow-up, 80% of the patients (12 with cervical and 4 with thoracic injuries), who initially presented with neurologically complete injury, recovered some degree of sensory or motor function; 20% (2 cervical and 2 thoracic) did not. Twelve patients (9 with cervical and 3 with thoracic injuries) recovered some sensorimotor function, while 3 with cervical injury and 1 thoracic injury recovered some sensation only. Two males regained the ability to walk. One, a patient who had sustained a cervical injury, could walk about 25 m unassisted. The other, a patient who had sustained a thoracic injury, regained the ability to walk 4 city blocks using canes. Two male patients with cervical injury could extend their legs against gravity but not enough to ambulate. Five patients could sense bladder fullness, and 3 males (2 with cervical injury and 1 with thoracic injury) could initiate voluntary micturition. Of 14 males, 7 recovered a subnormal voluntary erection, in 4 cases sufficient for limited sexual activity. Nine patients were able to attend school or university after the trauma (6 with cervical injury and 3 with thoracic injury) and 6 went back to work (3 with cervical injury and 3 with thoracic injury), 5 at professional occupations. Six patients (4 with cervical injury and 2 with thoracic injury) became self-sufficient.

We present follow-up results from the treatment of a series of 20 patients with complete cervical or thoracic SCI who underwent triple therapy with dexamethasone, surgical decompression, and local cord hypothermia. Although it is generally assumed that the majority of recovery has occurred by 2 years after complete SCI, we felt that a long-term follow-up assessment was indicated. Recent reports have indicated that conversion from ASIA Grade A impairment to Grade C may occur 3-5 years after the injury. Accordingly, it is prudent to follow up patients for at least 4-5 years to gauge their ultimate outcome.

This triple-therapy regimen appears to provide patients with substantial benefit compared with traditional forms of treatment. We compared data from the time period during which our study was conducted, which showed that about 32% of patients with complete cervical spine injuries would not survive 1 year post-injury and that about 5% could expect some degree of neurological improvement, with only 1% of patients with complete cervical SCIs recovering the ability to ambulate. In an effort to develop a predictive index for ambulatory outcomes after traumatic SCI, van Middendorp et al. found that having a complete spinal injury was itself associated with a negative predictive value of 92% with respect to being ambulatory at 1 year post-injury. Review of data collected from the Model Spinal Cord Injury System found that there was a 3.5% chance of status improving to ASIA Grade B and a 1.05% chance of improving to ASIA Grade C or D within 5 years of complete injury. An expert group reviewing data to provide information for the development of clinical trials on spinal cord injuries suggested a greater chance of functional improvement, concluding that 10% of patients with complete injury (ASIA Grade A) might convert to Grade B and 10% to Grade C within 1 year and less than 5% of patients with complete injury converting to ASIA Grade D. A larger proportion of the patients in our series achieved higher scores.

Of the 14 patients in our series with cervical SCIs, 4 (28.6%) were self-sufficient after recovery and 1 (7%) was able to ambulate. While data vary, more modern epidemiological studies have shown that, of persons with complete SCI, anywhere from 5% to 15% might expect to regain some degree of neurological function and from only 2% to 8% might expect to regain some degree of motor activity. Nine (64%) of our patients with cervical spinal injuries recovered some sensory and motor function, and 3 (21%) regained only some sensory function. Of particular interest is the patient in Case 4, who suffered a neurologically complete C-4 injury. According to data setting baseline expectations for Spinal Cord Independence measure III scores, persons with complete C-4 injury are unlikely to recover sufficient function to be functionally independent. The patient in Case 4 had such an injury and not only became independent but was also ambulatory following recovery. Data from a retrospective analysis indicate that about 3%-6% of persons with complete thoracic SCI might expect some degree of motor recovery. Three (50%) of the 6 patients in our series with thoracic SCI regained some degree of motor control, including 1 who became ambulatory. In fact, 2 patients (10%) in our series became ambulatory.

Respiratory problems were among the most common of the complications encountered in our series. This appears to be in concordance with literature on the subject: a 1994 study compiling 5 years of data found that 67% of acutely injured patients suffered some type of respiratory complication, namely atelectasis, pneumonia, or ventilatory failure. It has also been added that such injuries were more common with a more rostral spinal cord injury or more severe injury, an assessment verified in later literature. Decubitus ulceration unfortunately occurred in our series of patients. This might have been prevented in a setting where the patient is placed on a pressure-reduction mattress or routinely turned onto each side at least every 2 hours. Urinary tract complications were also very common, although intermittent catheterization was used throughout treatment. Such infections are frequent in patients with SCI. Other complications include gastrointestinal hemorrhage and vertebral instability.

At present, opinion on the use of glucocorticoids after SCI is divided, largely as a result of criticisms of the trials designed to examine their efficacy. In the years during which this series' patients were treated, glucocorticoids were considered a de facto standard of care. Given our personal experimental experience documenting the effects of these drugs, we felt they would be beneficial. One particular concern regarding their use is the risk of gastrointestinal bleeding, particularly given its intrinsic risk in spine-injured patients and epidemiological evidence associating steroids with gastrointestinal bleeding. In our series, 4 patients did experience gastrointestinal bleeding. One wonders whether this complication would have been as frequent had treatment taken place with modern stress ulcer prophylaxis.

The role of spinal cord decompression has also been questioned over the years. It is true that 5 of our patients were affected by some degree of orthopedic spinal instability after the initial operation, sometimes necessitating re-exploration and fusion; while this is a recognized complication of the decompressive surgery, it is of concern. Many researchers believe that immediate decompressive treatment after a severe SCI is indicated. There are clinicians who feel that the results of decompression are no better with early decompression (within 24 hours of injury) than with decompression days later. One review, however, showed that reduction of spinal fractures within 6 hours of injury allowed for the greatest degree of recovery. Other investigators have found that 26% of patients whose cord compression was reduced in the first 12 hours improved at least 2 grades on the Frankel Scale, while only 8% improved if the decompression was completed 12 hours following injury. Even late decompression may benefit some patients, suggesting that cord compression is an important component of neurological dysfunction. Newer data seem to agree that early, carefully performed decompression is indicated, particularly in situations where it can be performed soon after injury or in the presence of progressive neurological decline. There is still need for high-quality trials to examine the role of decompressive surgery in other, less obvious circumstances.

The final constituent of our treatment regimen was cooling. There has, over the years, been modest interest in therapeutic hypothermia for traumatic SCI, with interest more recently focusing on systemic cooling. Hypothermia has been shown to have beneficial effects in trauma as diverse as reducing tissue metabolic demand, decreasing hemorrhage and edema formation, moderating apoptosis and excitotoxicity, reducing glial cell activation, decreasing the strength of the inflammatory response, and preserving blood vessel and blood-brain barrier structure. Much modern research focuses on the therapeutic potential of mild systemic hypothermia (that is, a body temperature of 32°-35° C.). One particular technique, by which the body is cooled using closed-loop intravenous heat exchange, has been shown to be safe in a retrospective analysis and to yield promising results in a case-control study. By contrast, the technique used in the present series was regional hypothermia. Local cooling was initially valued because it avoided many of the potential complications of systemic hypothermia, notably arrhythmias, as well as potential long-term neurological deficits, which perhaps result from deranged cerebral blood flow regulation. In some circumstances, deeper cooling has been shown to be more effective than milder hypothermia. The ability to selectively deeply cool injured tissue while leaving core body temperature normal seems intuitively beneficial. Several series of cases seemed to indicate that regional hypothermia could be of great benefit. Regional cooling for the injured spinal cord, however, had fallen out of favor before high-quality controlled trials of its efficacy were conducted largely, it appears, because of criticisms of the requirement of a laminectomy, which might decrease spinal stability, and because of the supposedly lengthy period of time necessary to perform the procedure before cooling could be initiated. It is worth noting that, even if current expert opinion on decompression and fixation after acute SCI that surgical intervention should take place without delay were proven to be incorrect, a subset of patients, presumably those with significant cord compression and neurological deficits or progressive neurological deterioration, will require decompression regardless of whether cooling is instituted. Recent studies have suggested that it may be possible to perform local cooling without surgery. Even assuming that dural exposure is necessary, as it was in our patients, the average time to cooling in our series was about 7.1 hours after injury. Given that cooling was applied locally, the temperature in the area of interest decreased very rapidly to the target level, likely within 5 minutes. After a severe SCI, swelling increases for 3 to 5 days and then begins to recede. After only several hours of local profound cord cooling to a central cord temperature of about 17° C., the cord appeared to be less tight beneath the dura. This effect had not been observed after early decompression and steroid administration. This may indicate that low cord temperatures reduce posttraumatic cord swelling when hypothermia is initiated early. As a final note, after 1 patient with an incomplete severe cord lesion received local dural cooling at 6° C., he went on to have a remarkable recovery. These findings show that the procedure is unlikely to cause obvious harm. Therefore, sustained levels of deep regional hypothermia can be safely achieved by means of our epidural technique of heat exchange.

Given that an optimal neuro-protective temperature in traumatic SCI has not yet been established, we suggest the pursuit of a method that allows the safe and efficacious lowering of temperature in a targeted area of the body, particularly in cases in which decompressive surgery is indicated. One drawback of the current technique, and thus an area for potential improvement, is that we are unable to apply cooling directly after an SCI has been sustained. Should the injured tissue be rendered hypothermic at this early stage, substantial auto-destructive, secondary damage might be prevented. A method by which the spinal cord can be locally cooled to the desired temperature outside of hospitals would be of great value.

Due to the positive outcome enjoyed by a substantial number of our patients, it may be inferred that our treatment regimen could be beneficial. Given that this experimental treatment occurred in the context of a series of cases, rather than a clinical trial, it was not considered ethically appropriate to provide patients with less than what was, at the time, considered to be the best possible care—namely, local decompression and systemic glucocorticoid therapy.

We present here results of the treatment of 20 patients with neurologically-complete SCI by means of a combination of surgical decompression, glucocorticoid administration and regional hypothermia. These patients enjoyed better recovery than might have been expected with traditional forms of treatment. The benefit of steroid treatment for cord injury has been debated in the last decade but we feel that research into the effects of cord cooling should be expanded to include severe incomplete SCI. Given that the optimal neuroprotective temperature after acute trauma has not yet been defined, and may well be below that which is considered safely approachable through systemic cooling, methods that allow for the early attainment of such a temperature locally should be further explored. Any improvement in recovery of neurological function after such a devastating injury would be of great benefit to the victims of such injury. The cost savings to the health care system may also make a positive difference. The results are encouraging enough to suggest controlled clinical trials of treatment using localized spinal cord cooling, where such treatment can be instituted within hours following injury.

I claim:

1. A method of spinal cord cooling using posterior entry comprising the steps of:
a) exposing the spinal cord leaving the dura intact at the location of the spinal cord injury;
b) applying a cooling pad to the spinal cord dura at the point of the spinal cord injury in order to cool the dura which in turn cools the spinal cord;
c) cooling the pad to achieve a dura temperature of between 2 to 10 degrees centigrade for a period of ½ to 96 hours.

2. The method claimed in claim 1 wherein the cooling pad is a saddle shaped cooling saddle in order to follow the exterior profile of the dura to maximize the heat transfer between the saddle and the dura.

3. The method claimed in claim 2 wherein the cooling saddle is made of a light soft pliable material which conforms to the outer contour of the dura when gently placed on the dura.

4. The method claimed in claim 3 wherein the cooling saddle is made of a biomedical grade elastomer.

5. The method claimed in claim 2 wherein the cooling saddle includes an inlet and outlet for communicating cooling liquid through the pad thereby cooling the pad and the dura in contact with the pad.

6. The method claimed in claim 5 wherein the saddle includes channels for communicating cooling liquid through the saddle in a preselected path from the inlet to the outlet.

7. The method claimed in claim 1 wherein the saddle includes an inlet tube and an outlet tube each connected to a support nozzle for vertically gently suspending the saddle by the inlet and outlet tubes over the dura.

8. The method claimed in claim 1 wherein the cooling pad is a rounded quadrilateral in shape having a width of between 11 and 22 millimeters and a length of 11 to 34 millimeters.

9. The method claimed in claim 1 wherein the cooling pad includes a thermocouple for measuring the dura surface temperature and preferably a dural temperature of between 5 and 7 degrees centigrade is maintained for a period of 1 to 8 hours.

10. The method claimed in claim 1 wherein the inlet tube and the outlet tube attach to the cooling pad at an angle theta relative the pad to accommodate the spinal cord positioned on an inclined axis.

11. A method of spinal cord cooling using anterior entry comprising the steps of:
a) exposing the spinal cord using a tubular entry device leaving the dura intact at the location of the spinal cord injury;
b) applying a cooling pad to the spinal cord dura at the point of the spinal cord injury in order to cool the dura which in turn cools the spinal cord;
c) cooling the pad to achieve a dura temperature of between 2 to 10 degrees centigrade for a period of ½ to 96 hours.

12. The method claimed in claim 11 wherein the cooling pad is a round saddle shaped cooling saddle in order to fit through the tubular entry device and to follow the exterior profile of the dura to maximize the heat transfer between the saddle and the dura.

13. The method claimed in claim 12 wherein the cooling saddle is made of a light soft pliable material which conforms to the outer contour of the dura when gently placed on the dura.

14. The method claimed in claim 12 wherein the cooling saddle is made of a biomedical grade elastomer.

15. The method claimed in claim 12 wherein the cooling saddle includes an inlet and outlet for communicating cooling liquid through the pad thereby cooling the pad and the dura in contact with the pad.

16. The method claimed in claim 15 wherein the saddle includes channels for communicating cooling liquid through the saddle in a preselected path from the inlet to the outlet.

17. The method claimed in claim 11 wherein the cooling pad includes a thermocouple for measuring the dura surface temperature and preferably a dural temperature of between 5 and 7 degrees centigrade is maintained for a period of 1 to 8 hours.

18. A cooling system for cooling the spinal cord dura which in turn cools the spinal cord, the cooling system comprising;
a) a saddle shaped cooling saddle which can be lightly rested onto the exterior profile of the spinal dura to maximize the heat transfer between the saddle and the dura,
b) the cooling saddle includes an inlet and outlet and at least one channel for communicating cooling liquid through the saddle thereby cooling the saddle and the dura in contact with the saddle;

19. The cooling system claimed in claim 18 wherein the saddle includes an inlet tube and an outlet tube connected to the inlet and outlet respectively, for communicating cooling liquid to and from the saddle, each tube is connected to a support nozzle for vertically gently suspending the saddle by the inlet and outlet tubes over the dura.

20. The cooling system claimed in claim 19 further includes an articulating arm and a cooling unit, the articulating arm attached to the support nozzles and communicating cooling fluid between the support nozzles and the cooling unit, the cooling unit for cooling the cooling liquid to a preselected temperature.

21. The cooling system claimed in claim 18 wherein the cooling saddle is made of a light soft pliable material which conforms to the outer contour of the dura when gently placed on the dura.

22. The cooling system claimed in claim 18 wherein the cooling saddle is made of a biomedical grade elastomer.

23. The cooling system claimed in claim 18 wherein the cooling saddle is a rounded quadrilateral in shape having a width of between 11 and 22 millimeters and a length of 11 to 34 millimeters.

24. The cooling system claimed in claim 18 wherein the cooling saddle includes a thermocouple for measuring the dura surface temperature and preferably a dural temperature of between 5 and 7 degrees centigrade is maintained for a period of 1 to 8 hours.

25. The cooling system claimed in claim 19 wherein the inlet tube and the outlet tube attach to the cooling saddle at an angle theta relative the cooling saddle to accommodate the spinal cord positioned on an inclined axis.

26. The cooling system claimed in claim 21 wherein the cooling saddle is a round saddle shaped cooling saddle in order to fit through a tubular entry device and saddle shaped to follow the exterior profile of the dura to maximize the heat transfer between the saddle and the dura.

27. The cooling system claimed in claim 21 wherein the cooling saddle weighs between 1 and 6 grams to avoid damage to the spinal cord.

28. The cooling system claimed in claim 21 wherein the cooling saddle weighs between 2 and 4 grams to avoid damage to the spinal cord.
